(19) United States
(12) Patent Application Publication
(10) Pub. No.: US 2010/0172995 A1
(43) Pub. Date: Jul. 8, 2010

(54) Process For Preparing A Solid Pharmaceutical Composition

(75) Inventors: Iztok Klobcar, Zuzemberk (SI); Alesa Puncuh-Kolar, Smarjeske Toplice (SI); Anica Grandovec, Trebnje (SI); Urska Turk, Novo mesto (SI); Polona Solmajer-Lampic, Ljubljana (SI)

Correspondence Address: SWANSON & BRATSCHUN, L.L.C., 8210 SOUTHPARK TERRACE, LITTLETON, CO, 80120, US

(73) Assignee: LES LABORATOIRES SERVIER, Neuilly Sur Seine (FR)

(21) Appl. No.: 12/477,727

(22) Filed: Jun. 3, 2009

Related U.S. Application Data
(62) Division of application No. 10/599,154, filed on Jan. 11, 2007.

(30) Foreign Application Priority Data
Mar. 29, 2004 (DE) 1020040198454
Dec. 9, 2004 (DE) 1020040595216

(51) Int. Cl. A61K 31/403; A61K 9/14; A61K 31/404; A61P 9/12

(52) U.S. Cl. 424/489; 514/415; 514/412

(57) ABSTRACT

The invention relates to a process for preparing a solid pharmaceutical composition of perindopril or a salt thereof which avoids a wet granulation step and results in very stable pharmaceutical compositions, like tablets.

### RELATEDNESS OF THE APPLICATION

This application is a divisional application of co-pending U.S. Ser. No. 10/599,154, filed Jan. 11, 2007, which is a 35 U.S.C. §371 national phase application of PCT/EP2005/003277 (WO 2005/094793), filed on Mar. 29, 2005, entitled “Process for Preparing a Solid Pharmaceutical Composition,” which claims priority to German Application Serial No. 10 2004 019 845.4, filed Mar. 29, 2004, and German Application Serial No. 10 2004 059 521.6, filed Dec. 9, 2004. Each of these applications is specifically incorporated herein by reference in its entirety.

### BACKGROUND OF THE INVENTION

The invention relates to a process for preparing a solid pharmaceutical composition of perindopril or a salt thereof as well as a solid pharmaceutical composition.

ACE inhibitors, such as Perindopril, are a prodrug for perindoprilat which is in vivo the actually active substance. Especially solid formulations like tablets suffer from substantial degradation and thereby reduce the effective amount of perindopril. The main degradation routes are 1) the hydrolysis of the ester group and 2) intramolecular cyclization resulting in diketopiperazine (DKP), especially in an acidic environment.

There have been various attempts to stabilize solid compositions of ACE inhibitors.

According to EP-280 999 alkali or alkaline earth metal carbonates have been used to stabilize ACE inhibitor formulations. It is disclosed that in particular magnesium carbonate is a suitable stabilizing carbonate which proves to be effective when combined with enalpril. The components of the compositions are processed by means of wet granulation to the desired tablets.

Further WO 03/075842 discloses formulations of moexipril hydrochloride which have been stabilized by addition of alkali or alkaline earth metal carbonates. A mixture including moexipril hydrochloride as well as the alkaline reacting carbonate is processed by wet granulation so that the stabilizing effect is likely due to the in-situ forming of the sodium salt of moexipril. It is further disclosed that the amount of the carbonate should be greater than the stoichiometric amount of the moexipril hydrochloride.

In the same manner U.S. Pat. No. 5,350,582 discloses the use of stabilizing the ACE inhibitor enalapril maleate by addition of alkaline reacting substances which results in formation of the corresponding more stable sodium salt of enalapril. This in-situ reaction may be accomplished by using sodium hydrogen carbonate and use of a wet granulation process which allows the neutralization between the alkaline stabilizer and the enalapril maleate to occur. For 1 mole of enalapril maleate a total of 3 moles of sodium hydrogen carbonate are used.

However, the afore-mentioned approaches of obtaining stabilized formulations of ACE inhibitors, like perindopril, suffer from the drawback that they always include use of water which in turn can give rise to a reduced stability. Moreover, these processes often do not allow preparation of a pharmaceutical composition which shows a satisfactory level of stability, especially when stored over long periods of time. Finally, the use of a wet granulation step always requires means to remove the granulation liquid at a later stage in order to arrive at the final solid composition.

### DESCRIPTION OF THE INVENTION

It is therefore an object of the present invention to provide a process for preparing a solid pharmaceutical composition of perindopril which avoids the above problems of the conventional processes as well as a solid pharmaceutical composition of perindopril which has a high stability and contains only minor amounts of degradation products.

This object is surprisingly achieved by the process according to claims 1 to 12 and the composition according to claims 13 to 17.

The process according to the invention for preparing a solid pharmaceutical composition of perindopril or a salt thereof comprises
(i) dry mixing of perindopril or a salt thereof with at least one inorganic carbonate, at least one carrier, and optionally other components, and
(ii) dry processing of the mixture obtained in step (i) to the desired solid form.

In step (i) perindopril or a salt thereof is dry mixed with at least one inorganic carbonate, at least one carrier and optionally other components. The term “dry mixing” means that to none of the ingredients to be mixed a liquid, like water, ethanol or combinations thereof, is added and additionally that the mixing is effected without adding such a liquid.

Investigations have shown that the perindopril is preferably used in form of its tert.-butylamine salt, which is also referred to as perindopril erbumine, as this leads to particularly stable compositions.

Perindopril erbumine can exist in various polymorphic forms, for example form a disclosed in WO 01/87835, form β disclosed in WO 01/87836 and form γ disclosed in WO 01/83439. It is an advantage of the present composition that an undesired transformation of a polymorph is prevented or at least strongly reduced.

The inorganic carbonate is preferably sodium carbonate, sodium hydrogen carbonate, magnesium carbonate, calcium carbonate, calcium hydrogen carbonate or a mixture thereof.

It has further been shown that particularly stable compositions can be obtained when the molar ratio of perindopril or a salt thereof to inorganic carbonate is 1 to 0.1-0.9 and more preferably 1 to 0.05-0.83.

The carrier can be an inorganic or organic substance. Preferred examples of such carriers are dibasic calcium phosphate, tribasic calcium phosphate, magnesium oxide, microcrystalline cellulose, powdered cellulose, lactose and starch. In a more preferred embodiment the carrier is microcrystalline cellulose, lactose or a mixture thereof.

Particularly preferred is a microcrystalline cellulose which has a low moisture content of 0.3 to 5.0% by weight, preferably 0.3 to 1.5% by weight. The moisture content is determined as loss upon drying of a sample in a furnace at 100-150° C. until a constant mass is reached.

Additionally, the lactose is particularly preferably anhydrous lactose.

Compositions which have been obtained by using microcrystalline cellulose of the afore-mentioned low moisture content and/or anhydrous lactose show a very low level of degradation and are therefore highly stable products.

Optionally present other components are those conventionally used in the manufacture of pharmaceutical compositions and include for example disintegrants and lubricants. Preferred lubricants may be selected from the group consisting of magnesium stearate, calcium stearate, castor oil, glycerol monostearate, hydrogenated vegetable oil, polyethylene glycol, sodium stearyl fumarate, stearic acid, talc and zinc stearate.

It has further been found particularly preferred that the composition also comprises indapamide or a hydrate thereof. The water content of such a hydrate can vary depending on the humidity level of the atmosphere and can be up to 3% in case of the hemihydrate. A preferred hydrate is the hemihydrate.

It has also been shown that compositions are preferred which comprise indapamide or a hydrate thereof in form of particles having specific sizes. It is preferred that 90% by volume of the particles of indapamide or a hydrate thereof have a size of less than 80 μm, in particular of less than 70 μm.

These preferred particle sizes have a beneficial influence on content uniformity and the release profile of the composition.

In step (ii) the obtained mixture is dry processed to the desired solid form. The term “dry processing” means that no liquid is added to the mixture and that the processing is also effected without addition of any liquid. It is preferred that the mixture obtained in step (i) is processed by means of direct compression using a suitable apparatus, like a punch tableting machine.

Thus, the process according to the invention avoids the use of any liquids, including water or aqueous liquids, which on their own may lead to undesired degradation reactions. It is surprising that despite the avoiding of for example a wet granulation step it is possible by means of the process according to the invention to produce very stable compositions of perindopril or a salt thereof. In particular, it was found that tablets prepared according to the present process, after storage, form only small amounts of diketopiperazine (DKP). It is furthermore surprising that the use of small amounts of inorganic carbonate, i.e. below the stoichiometric amount, provide an additional stabilizing effect even though according to the prior art at least stoichiometric amounts need to be used.

The process according to the invention preferably results in tablets, minitablets or granules.

Further, the invention also relates to a solid pharmaceutical composition of perindopril or salt thereof, comprising
(a) perindopril or a salt thereof,
(b) at least one of microcrystalline cellulose having a moisture content of 0.3 to 5.0% by weight and anhydrous lactose,
(c) optionally at least one inorganic carbonate, and
(d) optionally other components.

The preferred embodiments of these composition have already been described above with respect to the process according to the invention. In such a preferred embodiment at least one inorganic carbonate is present in the composition according to the invention. In particular, it is preferred that the molar ratio of perindopril or a salt thereof to inorganic carbonate is 1 to 0.1-0.9 and preferably 1 to 0.50-0.83.

It is also preferred that the composition further comprises indapamide or a hydrate thereof. It is also preferred that 90% by volume of the particles of indapamide or a hydrate thereof have a size of less than 80 μm, in particular of less than 70 μm.

Moreover, the microcrystalline cellulose preferably has a moisture content of 0.3 to 1.5% weight.

It has surprisingly been shown that by using perindopril or a salt thereof in combination with either microcrystalline cellulose having the specified moisture content of 0.3 to 5.0% weight and/or anhydrous lactose a very stable composition is obtained. This is in contrast to the teaching of the prior art where the presence of liquid, such as water, is generally required for a wet granulation step or a neutralization reaction between alkaline stabilizer and acidic ACE inhibitor.

Additionally, the present process does not lead to a substantial transformation of polymorphs of perindopril or a salt thereof which is a further benefit in relation to conventional processes.

The following examples serve to illustrate the invention in more detail.

### EXAMPLES

### Example 1 (Comparison) and Examples 2 to 7 (Invention)

For the examples 2 to 4 (invention) perindopril erbumine as well as the materials used as carriers as well as the other components were screened. In examples 5 to 7 the preferred combination of perindopril erbumine with indapamide was used. The screened materials with the exception of the lubricant magnesium stearate were dry blended. Subsequently, the magnesium stearate was added to the resulting mixture and the mixture was homogenized. The homogenized mixture was then compressed using a punch tableting machine, Exacta X of Wilhelm Fette, to give tablets. As a comparison currently marketed tablets containing perindopril erbumine were used having the composition as given in the table below for example 1.

TABLE 1

| | Example | | | | | | |
|---|---|---|---|---|---|---|---|
| | 1 (comparison) | 2 | 3 | 4 | 5 | 6 | 7 |
| ingredient | mg/tablet | mg/tablet | mg/tablet | mg/tablet | mg/tablet | mg/tablet | mg/tablet |
| Perindopril erbumine | 4 | 4 | 4 | 4 | 4 | 4 | 4 |
| Indapamide | — | — | — | — | 1.25 | 1.25 | 1.25 |
| Microcrystalline | 22.50 | 22.50 | — | — | — | — | — |
| cellulose | | | | | | | |
| Microcrystalline | — | — | 22.50 | 22.50 | 22.50 | 22.50 | 22.50 |
| cellulose low | | | | | | | |
| moisture content | | | | | | | |
| of <1.5% | | | | | | | |
| Lactose monohydrate | 62.78 | 62.15 | — | 62.15 | 71.03 | 71.53 | 70.78 |
| Lactose anhydrous | — | — | 62.15 | — | — | — | — |
| Sodium hydrogen | — | 0.63 | 0.63 | — | 0.50 | — | 0.75 |
| carbonate | | | | | | | |
| Colloidal silica | 0.27 | 0.27 | 0.27 | 0.27 | 0.27 | 0.27 | 0.27 |
| Magnesium stearate | 0.45 | 0.45 | 0.45 | 0.45 | 0.45 | 0.45 | 0.45 |

* lactose anhydrous is lactose having a water content of less than 1% by weight, determined by the Karl-Fischer method according to Ph. Eur. 2.5.12.

The tablets prepared according to example 1 and according to examples 2, 3 and 4 were stored for 3 weeks at 50° C. in closed containers. The results given below show the amount of diketopiperazine after 3 weeks.

| | Diketopiperazine |
|---|---|
| Example | (DKP) (%) |
| 1 | 0.49 |
| 2 | 0.06 |
| 3 | 0.07 |
| 4 | 0.16 |

The comparison of example 1 with example 4 shows that even without alkaline reacting carbonate there is a decrease of the quantity of diketopiperazine in case of example 4 using microcrystalline cellulose of a low moisture content. In case of examples 2 and 3 even more decreased amounts of diketopiperazine were determined and these examples include as a stabilizer sodium hydrogen carbonate.

The amount of diketopiperazine was determined with a HPLC method using a Hypersil ODS column, 250 mm×4.6 mm i.d., packed with 5 μm particles, and a detector operating at a wavelength of 215 mm.

A gradient elution was effected using the following mobile phase
A: buffer solution of pH 2.0 prepared by adding into a 1000 ml volumetric flask, 0.92 g sodium heptansulfonate, and 1 ml Triethylamin (TEA) and filling with water to volume, and adjusting pH value of solution to 2.0 with perchloric acid
B: acetonitrile

| Time | | |
|---|---|---|
| (min) | % A | % B |
| 0 | 70 | 30 |
| 1 | 70 | 30 |
| 20 | 40 | 60 |
| 25 | 40 | 60 |
| 35 | 20 | 80 |
| 40 | 0 | 100 |
| 45 | 70 | 30 |

The flow rate of the mobile phase was set to 1.0 ml/min and the column temperature was set at 70° C. 20 μl of a standard solution and of the sample solution at a working concentration of about 3.0 mg/ml of perindopril erbumine in the buffer solution of pH 2.0 were injected. Diketopiperazine was detected on basis the of the retention time of the DKP peak on the chromatogram of the standard solution. The percentage of diketopiperazine was calculated as area %.

Tablets according to examples 2, 3 and 4 were additionally stored for 4 weeks at 40° C. in 75% relative humidity in closed containers. Again, the amounts of the degradation product diketopiperazine were determined as mentioned above and the results are given in the table below.

| | Diketopiperazine |
|---|---|
| Example | (DKP) (%) |
| 2 | 0.04 |
| 3 | 0.04 |
| 4 | 0.08 |

These experiments showed that a particularly stable composition was obtained in case of examples 2 and 3 which have been prepared without using any wet granulation step and which include sodium hydrogen carbonate as a stabilizer.

Thus, the above experiments show that by suitable selection of excipients and the avoiding of a wet granulation step tablets can be obtained which are, even in the presence of moisture, very stable against degradation.

1. Process for preparing a solid pharmaceutical composition of perindopril or a salt thereof, comprising
(i) dry mixing of perindopril or a salt thereof with at least one inorganic carbonate, at least one carrier, and optionally other components, and
(ii) dry processing of the mixture obtained in step (i) to the desired solid form.

2. Process according to claim 1, wherein the composition comprises the tert.-butyl amine salt of perindopril.

3. Process according to claim 1, wherein the inorganic carbonate is selected from the group consisting of sodium carbonate, sodium hydrogen carbonate, magnesium carbonate, calcium carbonate or calcium hydrogen carbonate.

4. Process according to claim 1, wherein the molar ratio of perindopril or a salt thereof to inorganic carbonate is 1 to 0.1-0.9 and preferably 1 to 0.50-0.83.

5. Process according to claim 1, wherein the carrier is microcrystalline cellulose, lactose or a mixture thereof.

6. Process according to claim 5, wherein the microcrystalline cellulose has a moisture content of 0.3 to 5.0% by weight, preferably 0.3 to 1.5% by weight.

7. Process according to claim 5, wherein the lactose is anhydrous lactose.

8. Process according to claim 1, wherein step (ii) is effected by direct compression of the mixture.

9. Process according to claim 1, wherein the composition also comprises indapamide or a hydrate thereof.

10. Process according to claim 9, wherein the hydrate is indapamide hemihydrate.

11. Process according to claim 9, wherein 90% of the particles of indapamide or a hydrate thereof have a size of less than 80 μm.

12. Process according to claim 11, wherein 90% of the particles of indapamide or a hydrate thereof have a size of less than 70 μm.
